(19) United States
(12) Patent Application Publication
(10) Pub. No.: US 2024/0066231 A1
(43) Pub. Date: Feb. 29, 2024

(54) Aspiration Syringe Device And Methods Of Use

(71) Applicants: Allen Smith, Knoxville, TN (US); Ryan Luttrell, Knoxville, TN (US); Quick Tube Medical, LLC, Knoxville, TN (US)

(72) Inventors: Allen Smith, Knoxville, TN (US); Ryan Luttrell, Knoxville, TN (US)

(21) Appl. No.: 18/503,653

(22) Filed: Nov. 7, 2023

Related U.S. Application Data
(63) Continuation of application No. PCT/US2022/028393, filed on May 9, 2022.
(60) Provisional application No. 63/185,983, filed on May 7, 2021.

(51) Int. Cl. A61M 5/31

(52) U.S. Cl. CPC A61M 5/3148; A61M 2005/3115

(57) ABSTRACT

The disclosure provides an aspiration syringe device and methods for use of such an aspiration syringe device. The aspiration syringe device includes an elongated housing, a plunger configured for axial sliding within the housing and being occludably received in the housing, a compression spring configured to urge the plunger away from the housing, and a retention mechanism configured to fixedly engage with the plunger so as to retain the plunger in position relative to the housing with the compression spring in an at least partially compressed state. The aspiration syringe device can be used in conjunction with another apparatus designed to penetrate a patient's body to access a distressed pleural space for the removal of air or fluid therefrom by allowing the air or fluid to flow in one direction from inside the body to outside the body.

### CROSS-REFERENCE TO RELATED APPLICATIONS

This patent application claims the benefit of U.S. Provisional Patent Application No. 63/185,983, entitled “Aspiration Syringe Device and Methods of Use,” filed on May 7, 2021, which is incorporated herein by reference in its entirety.

### FIELD

The disclosure relates to an aspiration syringe device and associated methods of using such an aspiration syringe device.

### BACKGROUND

Aspiration syringes generally have a volume of 40 mL or less. Such aspirations syringes may therefore be undesirable or time-consuming to use in certain applications (e.g., for chest drainage). Further, such aspiration syringes are not coupled with rapid deployment trochars. Such systems can also require separate suction sources and/or electrical sources that cannot be used for field use.

### SUMMARY

The aspiration syringe device of the disclosure may be implemented in a relatively small and inexpensive manner, permitting quick and effective usage of the device.

In an aspect, the aspiration syringe device includes a housing, a plunger, a spring, and a retention mechanism.

The housing may be an elongated housing. The housing may have an open proximal end. The housing may also have an open distal end.

The plunger may have a proximal end. The proximal end of the housing is occludably received through the proximal end of the housing. The plunger may also have a distal end. In one aspect, the plunger is configured for axial sliding within the housing.

The spring may be a compression spring. The spring may be disposed about a portion of the plunger external to the housing. In one aspect, the spring is configured to urge the distal end of the plunger away from the proximal end of the housing.

In one aspect, the retention mechanism is configured to fixedly engage with the plunger. The engagement between the retention mechanism and the plunger may retain the plunger in position relative to the housing. The engagement between the retention mechanism and the plunger may retain the spring in an at least partially compressed state.

In certain aspects, removal of the retention mechanism from engagement with the plunger may cause the compression spring to transition to an uncompressed state and urge the distal end of the plunger away from the proximal end of the housing.

In particular aspects, the retention mechanism may be configured to fixedly engage with the plunger adjacent the distal end of the plunger. The retention mechanism may pass through an opening defined in the housing. The opening may be defined in a side wall of the housing. The opening may also be defined adjacent the proximal end of the housing.

The retention mechanism may, in one aspect, be a pin. The pin may have a head and a shaft. The shaft may extend outwardly from the head. The shaft may be configured to be received through the opening defined in the housing. The shaft may also be configured to engage with the plunger.

In aspects, the compression spring may be disposed in circumferential relationship about the portion of the plunger external to the housing. The spring may also extend from the distal end of the plunger toward the proximal end of the plunger. The spring may have a first and a second end. The first end of the spring may abut a lateral extension at the proximal end of the housing. The second end of the compression spring may abut a flange portion at the distal end of the plunger.

The distal end of the housing may, in certain aspects, include a luer connector.

In particular aspects, the plunger may include a retention tab. The retention tab may project radially outward from the plunger. The retention tab may be configured to engage with the retention mechanism. Engagement between the retention tab and the retention mechanism may retain the plunger in position relative to the housing. Engagement between the retention tab and the retention mechanism may also retain the spring in the at least partially compressed state.

In certain aspects, the plunger may include a compressible stopper. The stopper may be positioned at the proximal end of the plunger. The stopper may be configured to provide a wiping action along an inner wall of the housing. The stopper may also be configured to prevent the plunger from being completely removed from the housing.

The housing may, in particular aspects, have a volume of at least 100 mL. In specific aspects, housing may have a volume of at least 300 mL.

In aspects, the spring may have a preselected tension configured to provide a desired suction pressure for the aspiration syringe device. In specific aspects, the spring may have a preselected tension configured to provide a suction pressure for the aspiration syringe device of from about −10 cmH2O to about −40 cmH2O.

Also provided herein is a method of determining a required amount of replacement blood for a mammalian patient using an aspiration syringe device as described herein. In an aspect, the method includes removing the retention mechanism from fixed engagement with the plunger, thereby causing the compression spring to transition to an uncompressed state and urge the distal end of the plunger away from the proximal end of the housing. In another aspect, the method further includes continuously removing blood contained within the mammalian patient as the compression spring transitions to the uncompressed state. In a further aspect, the method further includes determining an amount of blood removed from the mammalian patient by referencing an amount of blood contained within the housing after the compression spring has transitioned to the uncompressed state. In yet a further aspect, the method includes calculating a required amount of replacement blood as corresponding to the amount of removed blood contained within the housing.

In aspects, the method may further include replacing the blood removed from the mammalian patient with the calculated required amount of replacement blood.

Further provided herein is a method of measuring a volume of fluid loss during aspiration using an aspiration syringe device as described herein. In an aspect, the method includes removing the retention mechanism from fixed engagement with the plunger, thereby causing the compression spring to transition to an uncompressed state and urge the distal end of the plunger away from the proximal end of the housing. In another aspect, the method further includes continuously removing fluid contained within the mammalian patient as the compression spring transitions to the uncompressed state. In a further aspect, the method further includes determining an amount of fluid removed from the mammalian patient by referencing an amount of fluid contained within the housing after the compression spring has transitioned to the uncompressed state. In yet a further aspect, the method includes calculating a required amount of replacement fluid as corresponding to the amount of removed fluid contained within the housing.

In aspects, the method may further include replacing the fluid removed from the mammalian patient with the calculated required amount of replacement fluid. In aspects, the housing may have a volume of about 300 mL.

Also provided herein is a rapid deployment chest port. The rapid deployment chest port may, in aspects, include a frame, a removable plunger, a stabilizing component, and an aspiration syringe device as described herein.

The frame may include a lumen. The frame may also include a plunger port.

The removable plunger may define a bodily fluid inlet at a distal end thereof. The removable plunger may be within the lumen of the frame.

The stabilizing component may be configured to stabilize the frame inside and outside a chest cavity of a patient. The stabilizing component may further include a balloon attached to an outer diameter of the frame and an insertion stabilization platform attached to and slidable along the outer diameter of the frame proximal to the balloon.

The insertion stabilization platform may further comprise a fixation flexure. The fixation flexure may be operable to allow sliding of the insertion stabilization platform along the outer diameter of the frame. The fixation flexure may then be operable to secure the insertion stabilization platform to the frame.

The insertion stabilization platform may be no more than 7 cm away from the bodily fluid inlet when the rapid deployment chest port is inserted into the chest cavity of the patient.

The rapid deployment chest port may further comprise an external valve port. The external valve port may be attached to the outer diameter of the frame. The external valve port may be fluidly connected to the balloon. The external valve port may be a 1-way valve operable to receive a syringe to expand the balloon.

A ratio of a diameter of the balloon to a diameter of the frame may be 2.5 to 6.

A compressed expandable portion of the frame may be compressed by a peel away introducer. The peel away introducer may comprise finger grips. A bottom of the finger grips may be no more than 7 cm from the bodily fluid inlet.

The rapid deployment chest port may further comprise a handle. The handle may have a connector. The connector may be operable to removably attach the handle to the plunger port at a proximal end of the frame.

The rapid deployment chest port may further comprise an external check valve assembly. The external check valve assembly may be operable to connect to the plunger port and a suction source. The external check valve assembly may comprise a connector configured to connect to the plunger port, a valve outlet tubing operably associated with the connector, a check valve operably associated with the valve outlet tubing, and a valve inlet tubing operably connected to the check valve and proximal to the check valve.

A tip of the needle may comprise a concave curvature. The tip of the needle may have multiple concave curvatures.

The insertion stabilization platform may be operably connected to a pinch locking stabilizer configured to reversibly immobilize the insertion stabilization platform.

Additionally provided herein is a method of removing air or fluid contained within a pleural space of a mammalian patient using a rapid deployment chest port as described herein. In one aspect, the method includes inserting the bodily fluid inlet of the removable plunger and a distal portion of the frame of the rapid deployment chest port as described herein into a chest cavity of a patient. In an aspect, the method includes expanding the balloon on the inside of the chest cavity of the patient. In another aspect, the method includes removing the plunger from the plunger port. In a further aspect, the method includes connecting the aspiration syringe device to the plunger port. In yet a further aspect, the method includes removing air or fluid from the pleural space.

In aspects, the method may further include disconnecting the aspiration syringe device from the plunger port. In an additional aspect, the method may include expunging the removed air or fluid from the aspiration syringe device. In a further aspect, the method may include reconnecting the aspiration syringe device to the plunger port. In yet a further aspect, the method may include removing additional air or fluid from the pleural space. The method may further include determining a total amount of removed fluid and removed additional fluid. Further yet, the method may include calculating a required amount of replacement fluid as corresponding to the total amount of removed fluid and removed additional fluid. The method may further include replacing the fluid and additional fluid removed from the mammalian patient with the calculated required amount of replacement fluid.

Additional aspects and features are set forth in part in the description that follows, and will become apparent to those skilled in the art upon examination of the specification or may be learned by the practice of the disclosed subject matter. A further understanding of the nature and advantages of the disclosure may be realized by reference to the remaining portions of the specification and the drawings, which forms a part of this disclosure.

### BRIEF DESCRIPTION OF THE DRAWINGS

The accompanying drawing, that is incorporated in and constitutes a part of this specification, illustrates one variation of the disclosure and, together with the description, serves to explain the principles of the disclosure:

FIG. 1 illustrates a variation of an aspiration syringe device, according to illustrative embodiments.

FIG. 2 illustrates a variation of a rapid deployment chest port, according to illustrative embodiments.

FIG. 3 illustrates another variation of a rapid deployment chest port, according to illustrative embodiments.

FIG. 4 illustrates a variation of a method for using a rapid deployment chest port, according to illustrative embodiments.

FIG. 5 illustrates a variation of a needle having a concave curvature that can be disposed on a rapid deployment chest port, according to illustrative embodiments.

FIG. 6 illustrates a variation of a pinch locking stabilizer, according to illustrative embodiments.

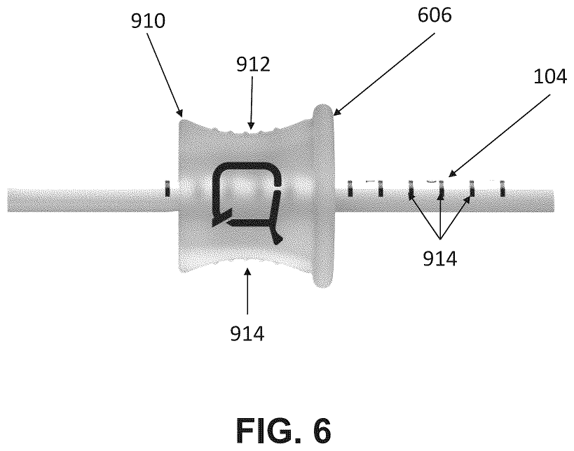

### DETAILED DESCRIPTION

In the following sections, detailed descriptions of examples and methods of the disclosure will be given. The description of both preferred and alternative examples are non-limiting, and it is understood that to those skilled in the art that variations, modifications, and alterations may be apparent. It is therefore to be understood that the examples do not limit the broadness of the aspects of the underlying disclosure as defined by the claims.

For purposes of this description, “distal” refers to the end extending into a body and “proximal” refers to the end extending out of the body.

For purposes of this description “connected to” includes two components being directly connected or indirectly connected with intervening components.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term.

The disclosure provides generally for syringe devices and methods for using the same. According to the disclosure, the syringe device may be used (e.g., as an aspiration syringe device) to remove air and/or fluid buildup, such as from within the pleural area of a patient, by moving a plunger backward (i.e., away from a housing within which a portion of the plunger is slidably disposed). The syringe devices of the disclosure allow for quick, standardized removal of air and/or fluid with a predetermined suction pressure through the use of urging means (e.g., a compression spring) that urges the plunger away from and/or out of the housing, which urging means can be provided with a predetermined and/or preselected tension corresponding to a desired suction pressure.

The syringe devices described herein may be particularly useful in certain applications (e.g., in-field use). By way of non-limiting example, in certain variations, the syringe devices may not be employed in a sterile setting, thus simplifying the manufacture and eventual use of the devices. Additionally, the syringe devices of the disclosure may, in certain variations, be used without requiring any complex mechanical or electrical components, thereby making such syringe devices particularly advantageous for certain applications (e.g., in-field use).

The disclosure also provides generally for methods and an apparatus for treating tension pneumothorax using a rapid deployment chest port. According to the disclosure, a rapid deployment chest port is inserted into the pleural area of a patient's body using a sharpened surface, such as a blade, needle, sharp tip, or knife edge. The sharpened surface may be attached to the rapid deployment chest port. Following insertion, the rapid deployment chest port may be expanded to open a cavity to relieve pressure from air and/or fluid buildup within the pleural space. In some variations, the rapid deployment chest port may further use suction to remove fluid from the pleural space.

The rapid deployment chest port allows for quick, standardized insertion of a chest tube without requiring creating an incision with a scalpel prior to insertion, as is current practice. Making an incision with a scalpel leads to inconsistent incisions that may be too large for the chest port, such that there may be an open wound around the chest port that may require suturing. Thus, the rapid deployment chest port provides less risk for infection in the patient because it creates a standardized incision that is the exact size needed for the rapid deployment chest port. In addition, a standard chest port requires suturing to stabilize the chest port so that it does not migrate within the patient. This requires additional time in the placement of the chest tube before the patient may be treated. The separate incision and suturing may lead to standard chest tubes taking several minutes to be inserted and ready for use. Because the rapid deployment chest port does not require a separate incision or any additional suturing, it provides for a reduction in the amount of time to insert the chest port and begin treating the patient. In some variations, the rapid deployment chest port may be deployed within 20 seconds. In some variations, the rapid deployment chest port may be deployed within 30 seconds. In some variations, the rapid deployment chest port may be deployed within 60 seconds. In some variations, the rapid deployment chest port may be deployed within 90 seconds.

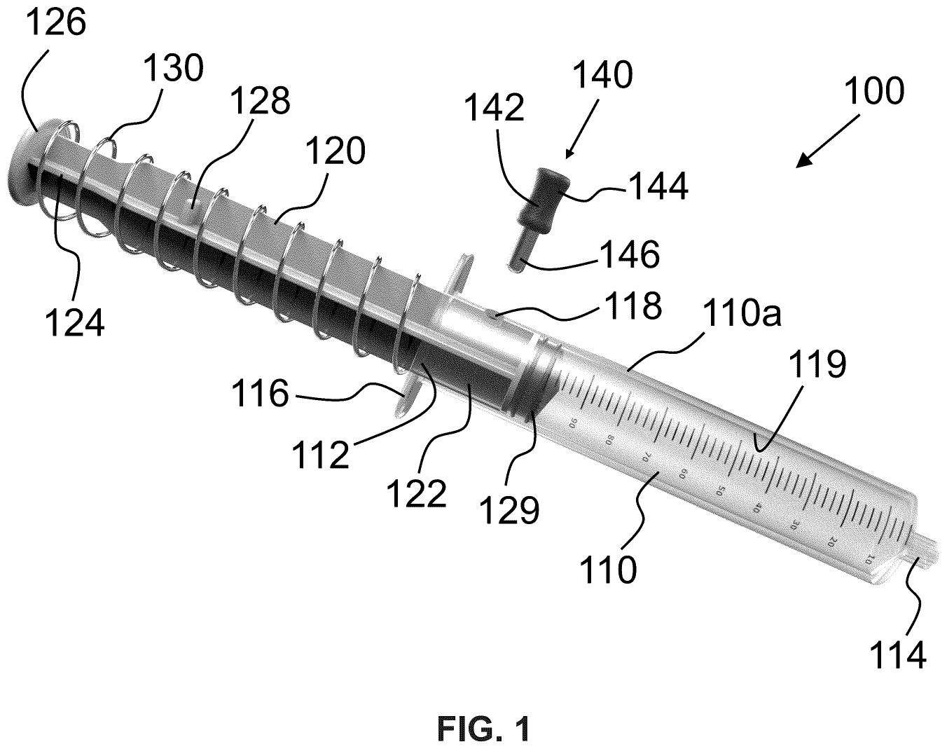

Referring now to FIG. 1, an example variation of the aspiration syringe device 100 is illustrated. FIG. 1 illustrates the aspiration syringe device 100 in an uncompressed state. The aspiration syringe device 100 may include a housing 110. As illustrated in FIG. 1, the housing may be an elongated housing 110 generally extending between a distal end 114 and a proximal end 112 thereof. In variations, the housing 110 may be defined by a side wall 110a. The side wall 110a may extend from the proximal end 112 of the housing 110 to the distal end 114 thereof. The side wall 110a may define an inner wall 119 of the housing. In variations, the side wall 110a of the housing 110 may be at least partially transparent so as to provide visual indication to a user of any fluid contained within the housing. Additionally, in certain variations, the side wall 110a of the housing 110 may include one or more indicia at predetermined intervals and/or locations along the side wall so as to provide visual indication to a user of the level of any fluid contained within the housing (e.g., so that the user may quickly and effectively identify the volume of fluid contained within the housing).

The distal end 114 of the housing may be an open distal end. In example variations, the open distal end 114 of the housing 110 may be configured as an inlet, an outlet, or both. The open distal end 114 of the housing 110 may be configured to receive and/or expel air, fluid, or both therethrough. In variations, the open distal end 114 of the housing 110 may include a luer connector. In such variations, the aspiration syringe device 100 may generally be configured to be attachable or otherwise in fluid communication with another device (e.g., another device having a complementary luer connector) at the open distal end 114 of the housing 110.

The proximal end 112 of the housing may be an open proximal end. In example variations, the open proximal end 112 of the housing 110 may be configured to receive at least a portion of a plunger 120 therethrough. In variations, the housing 110 may include a lateral extension 116. The lateral extension 116 may be positioned at the proximal end 112 of the housing 110. The lateral extension 116 may generally project outwardly from the side wall 110a of the housing 110.

The housing 110 may be of any desired size and/or shape to suit a particular application. By way of non-limiting example, the housing 100 may have a volume of from about 5 mL to 500 mL or greater. In some variations, the housing 110 may have a volume of at least 5 mL. In some variations, the housing 110 may have a volume of at least 25 mL. In some variations, the housing 110 may have a volume of at least 50 mL. In some variations, the housing 110 may have a volume of at least 75 mL. In some variations, the housing 110 may have a volume of at least 100 mL. In some variations, the housing 110 may have a volume of at least 125 mL. In some variations, the housing 110 may have a volume of at least 150 mL. In some variations, the housing 110 may have a volume of at least 175 mL. In some variations, the housing 110 may have a volume of at least 200 mL. In some variations, the housing 110 may have a volume of at least 225 mL. In some variations, the housing 110 may have a volume of at least 250 mL. In some variations, the housing 110 may have a volume of at least 275 mL. In some variations, the housing 110 may have a volume of at least 300 mL. In particular applications (e.g., removal of blood), a volume of at least 300 mL may be particularly advantageous as the volume of the housing may generally equate to a pint of blood and may be used to determine an amount of blood removed using the aspiration device 100 and/or a required amount of replacement blood, as described herein. By way of non-limiting example, the housing may generally be hollow and cylindrical. By way of further non-limiting example, the housing 110 may generally be made from any suitable material.

As described above, the plunger 120 may be configured to be at least partially received through the open proximal end 112 of the housing 110. As illustrated in FIG. 1, the plunger 110 may generally extend between a distal end 124 and a proximal end 122 thereof. The proximal end 122 of the plunger 120 may be occludably received through the proximal end 112 of the housing 110. In this way, fluid within the housing 110 may generally be prevented or retarded from exiting the housing 110 through the proximal end 112 thereof.

In variations, the plunger 120 may be configured for axial sliding within the housing 110. Put another way, the plunger 120 may be configured for selective telescoping motion in and out of the housing 110. In variations, the plunger 120 may be slid axially within the housing between (a) a first position in which the proximal end 122 of the plunger 120 is positioned adjacent to the distal end 114 of the housing 110 and the distal end 124 of the plunger 120 is positioned adjacent to the proximal end 112 of the housing 110 and (b) a second position in which the proximal end 122 of the plunger 120 is spaced apart from the distal end 114 of the housing 110 and positioned adjacent to the proximal end 112 of the housing 110 and the distal end 124 of the plunger 120 is spaced apart from the proximal end 112 of the housing 110. As will be appreciated, the plunger 120 may further be positioned in a variety of other intermediate positions between the first and second positions, as desired.

In variations, the plunger 120 may include a stopper 129. The stopper may be a compressible stopper 129. The compressible stopper 129 may be positioned at the proximal end 122 of the plunger 120. The compressible stopper 129 may be configured to provide a wiping action along the inner wall 129 of the housing 110. Additionally, the compressible stopper 129 may be configured to prevent the plunger 120 from being completely removed from the housing 110, such as by occludably interfacing with the proximal end 112 of the housing 110. In variations, the plunger 110 may include a flange portion 126. The flange portion 126 may be positioned at the distal end 124 of the plunger 120. The flange portion 126 may project outwardly from a main body portion of the plunger 120.

The plunger 120 may be of any desired size and/or shape to suit a particular application. By way of non-limiting example, the plunger 120 may be sized and/or shaped to generally correspond to the size and/or shape of the housing 110. By way of non-limiting example, the plunger 120 may generally be made from any suitable material.

The syringe device 100 further includes a biasing means. In the variation illustrated in FIG. 1, the biasing means are in the form of a spring 130. The spring 130 may generally be configured to urge the plunger 120 away from and/or out of the housing 110. Put another way, the spring 130 may generally be configured to urge the plunger 120 through the interior of the housing 110 in a distal direction D. As such, the spring may be in the form of a compression spring 130. In the variation illustrated in FIG. 1, the spring 130 is in the uncompressed state such that the spring 130 has urged the plunger 120 away from and/or out of the housing 110 (e.g., to the second position described above). The spring 130 may generally be configured to urge the distal and 124 of the plunger 120 away from the proximal end 112 of the housing 110. Conversely, the distal and 124 of the plunger 120 may be slid back toward the proximal end 112 of the housing 110 (e.g., to the second position described above) by overcoming the biasing force of the spring 130 (e.g., by a user pressing against the distal end 124 of the plunger 120). As will be appreciated, when the spring 130 is in the compressed state, a majority of the plunger 120 may be disposed and/or received within the housing 110. Conversely, when the spring 130 is in the uncompressed state, a majority of the plunger 120 may be external to the housing 110 due to the plunger being urged away from and/or out of the housing 110 by the spring 130 (refer to FIG. 1). In variations, the spring 130 may be configured to continuously urge the plunger 120 away from and/or out of the housing 110 at a predetermined rate so as to provide safe yet continuous aspiration of fluid.

In variations, the spring 130 may be disposed about the plunger 120. The spring 130 may, in certain variations, be disposed about a portion of the plunger 120 external to the housing 110. As illustrated in the variation of FIG. 1, the spring 130 may be disposed in circumferential relationship about the portion of the plunger 120 external to the housing 110. Put another way, the spring 130 may encircle and/or surround at least a portion of the plunger 120 external to the housing 110.

In variations, the spring 130 may extend from the distal end 124 of the plunger 120 toward the proximal end 122 thereof. The spring 130 may have a first end and an opposite second end. The spring 130 may generally extend from the first end thereof to the second end thereof. The first end of the spring 130 may be positioned adjacent the proximal end 112 of the housing 110. The first end of the spring 130 may, in certain variations, abut the lateral extension 116 at the proximal end 112 of the housing 110. The second end of the spring 130 may be positioned adjacent the distal end 124 of the plunger 120. The second end of the spring 130 may, in certain variations, abut the flange portion 126 at the distal end 124 of the plunger 120.

The spring 130 may be of any desired size and/or shape to suit a particular application. By way of non-limiting example, the spring 130 may be sized and/or shaped to generally correspond to the size and/or shape of the plunger 120. In variations, the spring 130 may be of a predetermined length when in the uncompressed state that corresponds to a length of the plunger 120 such that the plunger 120 cannot be urged completely out of the housing 110 by the urging force of the spring 130. By way of non-limiting example, the spring 130 may generally be made from any suitable material.

The spring 130 may have any desired tension to suit a particular application. In variations, the spring 130 may have a preselected tension. The spring 130 may have a preselected tension configured to provide a desired suction pressure for the syringe device 100. By way of non-limiting example, the spring 130 may have a preselected tension configured to provide a desired suction pressure for the syringe device 100 of from about −10 to about −40 cmH2O. In some variations, the spring 130 may have a preselected tension configured to provide a desired suction pressure for the syringe device 100 of −10 cmH2O. In some variations, the spring 130 may have a preselected tension configured to provide a desired suction pressure for the syringe device 100 of −15 cmH2O. In some variations, the spring 130 may have a preselected tension configured to provide a desired suction pressure for the syringe device 100 of −20 cmH2O. In some variations, the spring 130 may have a preselected tension configured to provide a desired suction pressure for the syringe device 100 of −30 cmH2O. In some variations, the spring 130 may have a preselected tension configured to provide a desired suction pressure for the syringe device 100 of −40 cmH2O. In certain applications, it may be desirable to for the spring 130 to have a preselected tension configured to provide a desired suction pressure for the syringe device 100 of less than or equal to −40 cmH2O so as to minimize the risk of any undesirable aspiration (e.g., aspirating tissue and/or spongy organs).

The syringe device 100 further includes a retention mechanism 140. The retention mechanism may generally be configured to retain the plunger 120 in position relative to the housing 110. In variations, the retention mechanism may be configured to retain the plunger 120 at a predetermined position relative to the housing 110. The retention mechanism 140 may, in certain variations, fixedly engage with the plunger 120. The retention mechanism 140 be configured to prevent sliding and/or telescoping motion of the plunger 120 within the housing 110 by fixedly engaging the plunger 120. The retention mechanism 140 may, in certain variations, be configured to retain the plunger 120 in position relative to the housing 110 with the spring 130 in an at least partially compressed state. Conversely, as will be appreciated, the removal of the retention mechanism 140 from fixed engagement with the plunger 120 may cause the spring 130 to transition to an uncompressed state, thereby urging the plunger 120 away from and/or out of the housing 110 as described herein.

In the variation illustrated in FIG. 1, the retention mechanism 140 may be configured to engage (e.g., fixedly engage) with the plunger 120 adjacent the distal end 124 thereof. In variations, the retention mechanism 140 may pass through an opening 118 defined in the housing 110. The opening 118 may be defined in the side wall 110a of the housing 110. In certain variations, the opening 118 may be defined adjacent the proximal end 112 of the housing 110.

In certain variations, the plunger 120 may include retention tab 128, such as is illustrated in FIG. 1. The retention tab 128 may project outwardly from a main body portion of the plunger 120. The retention tab 128 may generally be configured to engage with the retention mechanism 140 so as to retain the plunger 120 in position relative to the housing 110 with the spring 130 in the at least partially compressed state.

In the variation illustrated in FIG. 1, the retention mechanism 140 is a grenade pin 142, though other variations are not so limited. In variations the retention mechanism 140 in the form of a grenade pin 142 may have a head 144 and a shaft 146. The shaft 146 may extend outwardly from the head 142. The shaft 146 may be configured to be received through the opening 118 defined in the housing 110. The shaft 146 may be configured to engage with the plunger 120. In certain variations, the shaft 146 may be configured to fixedly engage with the retention tab 128 of the plunger 120. In other variations, the retention mechanism 140 could be a clickable button or any other structure capable of fixedly engaging with the plunger 120 so as to retain the plunger 120 in position relative to the housing 110 with the spring 130 in the at least partially compressed state.

The retention mechanism 140 may be of any desired size and/or shape to suit a particular application. By way of non-limiting example, the retention mechanism 140 may generally be made from any suitable material.

The syringe device 100 as described herein may be used in a variety of applications. By way of non-limiting example, the syringe device 100 may be employed in (a) a method of determining a required amount of replacement blood for a mammalian patient, (b) a method of treating tension pneumothorax, and/or (c) a method of measuring of measuring a volume of fluid loss during aspiration. Moreover, the syringe device 100 as described herein may be used as a component of various other devices. By way of non-limiting example, the syringe device 100 may be employed as part of a rapid deployment chest port.

In an example method of determining a required amount of replacement blood for a mammalian patient, a syringe device 100 as described herein may be employed. The retention mechanism 140 may be removed from fixed engagement with the plunger 120, thereby causing the spring 130 to transition to an uncompressed state and urge the plunger 120 away from and/or out of housing 110 as described herein. Blood contained within the mammalian patient may be continuously removed as the spring 130 transitions to the uncompressed state (e.g., and urges the plunger 120 away from and/or out of the housing 110, thereby creating a suction pressure permitting the blood to be received from the mammalian patient and into the housing). In variations, the housing 110 may have a volume of about 300 mL, generally corresponding to a pint of blood. In such variations, once the spring 130 is in the uncompressed state and the housing 110 has filled with blood removed from the mammalian patient, a user of the syringe device 100 may quickly and reliably understand that a pint of blood has been removed from the mammalian patient (and, consequently, that a pint of blood should be replaced). In variations, the amount of blood removed from the mammalian patient may be determined by referencing the amount of blood contained within the housing after the spring 130 has transitioned to the uncompressed state. The required amount of replacement blood may be calculated as corresponding to the amount of removed blood contained within the housing 110. The blood removed from the mammalian patient may then be replaced with the calculated required amount of replacement blood.

In an example method of measuring a volume of fluid loss during aspiration, a syringe device 100 as described herein may be employed. The retention mechanism 140 may be removed from fixed engagement with the plunger 120, thereby causing the spring 130 to transition to an uncompressed state and urge the plunger 120 away from and/or out of housing 110 as described herein. Fluid contained within the mammalian patient may be continuously removed as the spring 130 transitions to the uncompressed state (e.g., and urges the plunger 120 away from and/or out of the housing 110, thereby creating a suction pressure permitting the fluid to be received from the mammalian patient and into the housing). In variations, the housing 110 may have a volume of about 300 mL, generally corresponding to a pint of fluid. In such variations, once the spring 130 is in the uncompressed state and the housing 110 has filled with fluid removed from the mammalian patient, a user of the syringe device 100 may quickly and reliably understand that a pint of fluid has been removed from the mammalian patient (and, consequently, that a pint of fluid should be replaced). In variations, the volume of fluid loss during removal from the mammalian patient may be determined by referencing the volume of fluid contained within the housing after the spring 130 has transitioned to the uncompressed state. The required amount of replacement fluid may be calculated as corresponding to the volume of removed fluid contained within the housing 110. The fluid removed from the mammalian patient may then be replaced with the calculated required amount of replacement fluid.

The foregoing method of determining a required amount of replacement blood for a mammalian patient and/or method of measuring a volume of fluid loss during aspiration may be particularly advantageous in certain applications (e.g., in-field use). By way of non-limiting example, use of the syringe device 100 as described herein in either of the foregoing methods may provide a quick, efficient, and accurate means by which a user can know exactly how much blood or fluid necessary to be replaced, which can be particularly useful in combat.

In examples, the syringe device of the disclosure can be employed as part of a rapid deployment chest port. A suitable rapid deployment chest port is shown and described in International Publication Number WO 2020/113159 A1, published Jun. 4, 2020, which is hereby incorporated by reference herein in its entirety for all purposes.

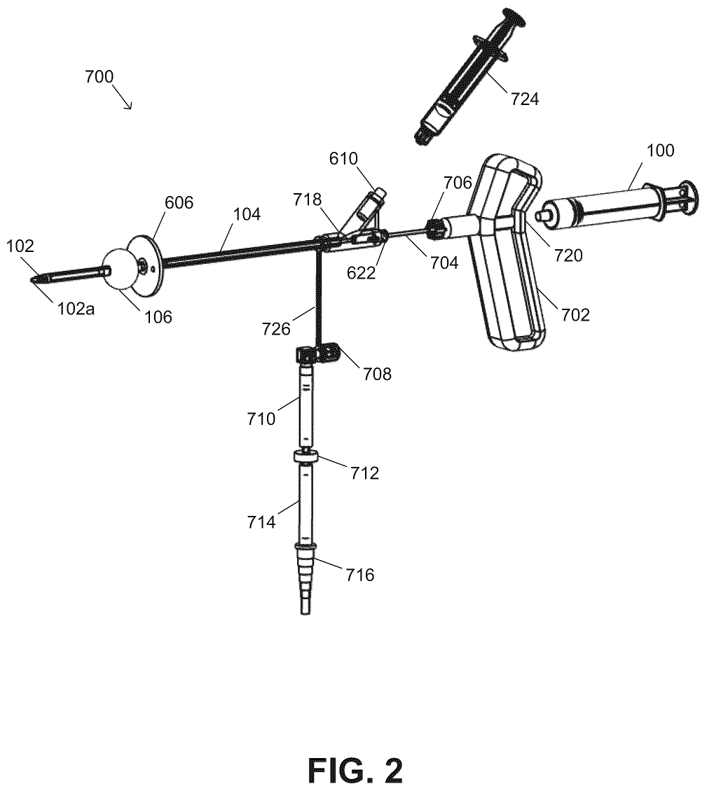

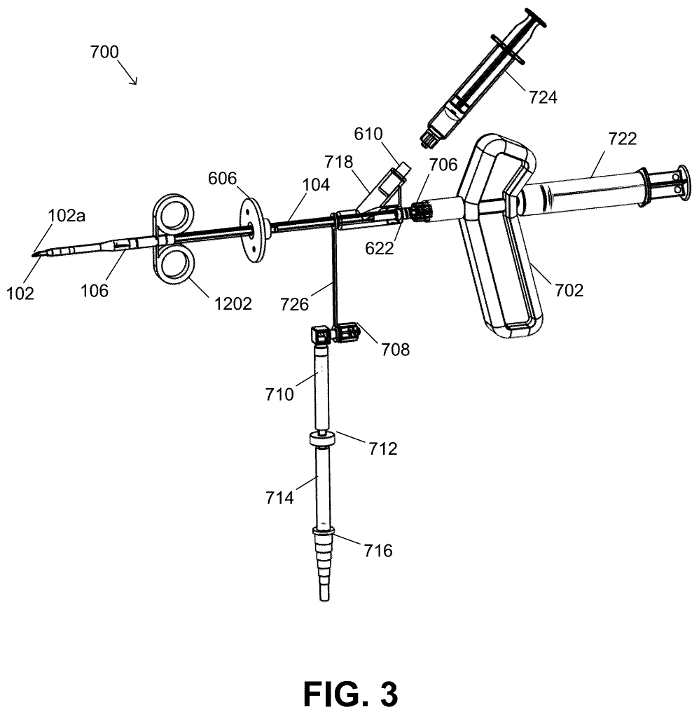

Referring now to FIG. 2, a variation of a rapid deployment chest port is shown. In some variations, the rapid deployment chest port 700, as illustrated, may be modular. By way of non-limiting example, the rapid deployment chest port 700 may include a plunger 704 linking a handle 702 (e.g., finger grips) to a needle 102 (which can include a blade, or a beveled needle) and/or to a bodily fluid inlet 102a (which may be an opening, a vent, or an aperture), a frame 104 (comprising a lumen for the plunger 704 and air and/or fluid, a Y-hub with an external valve port 610 and, in some variations, a plunger port 622 with a luer connector at the proximal end); and a stabilization component. In some variations, the stabilization component may include a balloon 106 and an insertion stabilization platform 606. In a variation, the plunger 704 may be a stylet shaft extending the length of the frame. In some variations, the needle 102 may be secured to the frame 104 or the distal end of the plunger 704. In some variations the needle 102 includes a sharp protrusion at a bottom of the rapid deployment chest port 700. The needle 102 includes a sharpened surface and non-limiting examples of the needle include a knife, a blade, a scalpel, a double-bladed scalpel, or other object with a surface of sufficient sharpness to penetrate through the thorax into the pleural space. In exemplary variations, the needle 102 is pointed, allowing the desired blunt dissection with minimal effect on the exterior of the patient's body. In some variations, the needle 102 may be blunt, such as in a cone shape, as seen in FIG. 3. In some variations, the needle 102 may be angled or curved, such as the point of a fountain pen, to naturally guide the needle over the intended rib, as seen in FIG. 3. The needle may be realized with or without an internal lumen. In variations including the lumen, the lumen may be used in conjunction with a syringe or other air-tight device to produce a vacuum while the device is advanced through the patient's tissue. For example, the needle may be fluidly connected to the frame and/or handle. The needle is attached to the distal end of the rapid deployment chest port such that it may penetrate through the patient to the pleural space without the need for a separate scalpel. This allows for a more precise incision that is sized for the rapid deployment chest port without creating a wider than necessary opening, which is often the case with a scalpel.

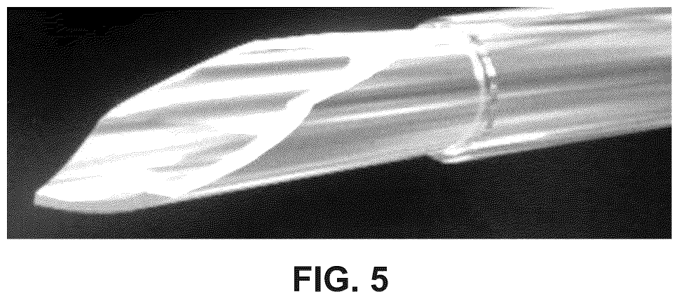

In some variations, the needle 102 has a concave curvature as depicted in FIG. 5. The needle has a concave shape from the tip to the end of the opening. In some variations, the needle can have multiple concave curvatures, as depicted in FIG. 5.

In some variations, the concave tip has a single concave tip. In some variations, the concave curvature can be configured to be greater than or equal to 10 degrees. In some variations, the concave curvature can be configured to be greater than or equal to 15 degrees. In some variations, the concave curvature can be configured to be greater than or equal to 20 degrees. In some variations, the concave curvature can be configured to be greater than or equal to 25 degrees. In some variations, the concave curvature can be configured to be greater than or equal to 30 degrees. In some variations, the concave curvature can be configured to be greater than or equal to 35 degrees. In some variations, the concave curvature can be configured to be greater than 40 degrees. In some variations, the concave curvature can be configured to be less than or equal to 45 degrees. In some variations, the concave curvature can be configured to be less than or equal to 40 degrees. In some variations, the concave curvature can be configured to be less than or equal to 35 degrees. In some variations, the concave curvature can be configured to be less than or equal to 30 degrees. In some variations, the concave curvature can be configured to be less than or equal to 25 degrees. In some variations, the concave curvature can be configured to be less than or equal to 20 degrees. In some variations, the concave curvature can be configured to be less than or equal to 15 degrees. Multiple concave curvatures can be formed in the tip, each with the same or different concave curvatures as other concave curvatures.

In some variations, when the rapid deployment chest port 100 is in an expanded state, the needle 102 may retract into the frame 104 at a needle retraction slot. The needle retraction slot may be any means for ensuring that the distal end (e.g., a pointed end) of the needle 102 is not exposed to the inside of the patient's body after retraction. In some variations, the needle retraction slot may comprise a means for wiping the tip of the needle 102. Such means may include, for example, a narrow slot opening or an absorbent membrane.

In variations, the needle 102 may include or define the bodily fluid inlet 102a. In other variations, the bodily fluid inlet 102a may be defined by or secured to the frame 104 or the distal end of the plunger 704. The bodily fluid inlet 102a may be fluidly connected to the frame and/or handle. Upon insertion of the rapid deployment chest port, the rapid deployment chest port may be used to relieve pressure from air and/or fluid buildup within the pleural space through the bodily fluid inlet 102a. In some variations, the rapid deployment chest port may further use suction to remove fluid from the pleural space, such as by employing the aspiration syringe device described herein. In other variations, increased pressure within the pleural space may cause air or fluid to move through the frame without use of a suction source.

Frame 104 may be comprised of any suitable material, such as plastic or steel. In other variations, the frame 104 may be substantially cylindrical. In additional examples, the frame may further include a peel away introducer at its distal end. In some specific examples, a frame 104 may be roughly pentagonal in shape, with one or more appendages extending from the point of the pentagon. Other shapes are within the scope of the invention.

In some variations, the frame 104 may be compliant, such that it may be compressed. In additional variations, the frame 104 may be a catheter, such as a silicone catheter, or a thermo plastic or rubber extrusion. In some variations, the frame may include a lip to aid in the insertion of the rapid deployment chest port 700, such that the frame does not collapse during insertion. In other variations, the frame may not include a lip when a peel away introducer is used to reinforce the frame during insertion. In addition, the use of an introducer may compress the outer diameter of the frame at the site of insertion. Thus, in some examples, the diameter of the frame may depend on the use of an introducer. In a variation, the frame may have a diameter ranging from 5 French to 40 French. In some variations, the frame may have a diameter of 5 French. In some variations, the frame may have a diameter of 8 French. In some variations, the frame may have a diameter of 10 French. In some variations, the frame may have a diameter of 16 French. In some variations, the frame may have a diameter of 20 French. In some variations, the frame may have a diameter of 25 French. In some variations, the frame may have a diameter of 30 French. In some variations, the frame may have a diameter of 35 French. In some variations, the frame may have a diameter of 40 French.

In this variation, the plunger 704 is connected to the needle 102 and/or the bodily fluid inlet 102a at the distal end and connected to finger grips at the proximal end. In other variations, the needle 102 and/or the bodily fluid inlet 102a may be integrated with the plunger 704, such that they are a single element. For example, the plunger 704 may have a tapered distal end, forming a needle and/or bodily fluid inlet. In other examples, the plunger 704 may terminate in a needle and/or bodily fluid inlet. In some variations, the needle 102 may be any structure capable of piercing the skin and penetrating through the body to the pleural space, and the bodily fluid inlet 102a may be any structure capable of permitting fluid and/or air to pass therethrough. Non-limiting examples of needles include a blade, sharp tip, and/or or knife edge, and non-limiting examples of bodily fluid inlets include an opening, vent, and/or aperture. In at least one example, the needle 102 may be a sharp silicone tip. In some variations, the finger grips may be a handle 702. The handle 702 may have an upper and lower portion, and the lower portion may be longer than the upper portion. In some variations, the upper and lower portions may be angled to provide an ergonomic handle. In some variations, the handle 702 may assist the user in guiding the rapid deployment chest port 700 to the desired spot. In a variation, the handle 702 may further include a syringe port 720 at the proximal end of the handle. In an example, the aspiration syringe device 100 may be attached to the syringe port 720, such as for testing the placement of the rapid deployment chest port 700. In this example, a user may withdraw the aspiration syringe device 100 to identify the fluid and/or air located at the needle 102, the bodily fluid inlet 102a, and/or the distal end of the frame 704. If the rapid deployment chest port 700 is in the incorrect location, the user may adjust the placement by moving the handle 702 towards or away from the patient, as appropriate. The aspiration syringe device 100 may again be used to test the placement of the rapid deployment chest port 700. The aspiration syringe 100 may be removed from the syringe port 720 on the handle once the correct placement of the rapid deployment chest port 700 is confirmed.

In some variations, the plunger structure may be removable from the frame 104. The plunger 704 may be inserted into the frame 104 through a plunger port 622 on the frame 104. In some variations, the plunger port 622 may be on a Y-hub 718. The plunger 704 may pass through a lumen in the frame and end in the needle 102 and/or the bodily fluid inlet 102a. In some variations, the plunger port 622 may include a luer connector. In some variations, the handle may include a reciprocal luer connector 706 to connect the handle to the plunger port 622. The plunger 704 may be already inserted, and then removed. For example, the plunger 704 may be removed from the frame 104 when the rapid deployment chest port 700 is placed in the pleural space of the patient. In some variations, when the plunger 704 is removed, a reciprocal luer connector 708 may connect to the plunger port 622 to attach an external check valve assembly to the frame 104. The external check valve assembly may operate to allow a check valve to be inserted into frame 104 without the check valve needing to be integrated into rapid deployment chest port 700. In some variations, the check valve assembly may include the luer connector 708, a check valve 712, valve outlet tubing 710 connected to the luer connector and distal to the check valve, valve inlet tubing 714 proximal to the check valve, and a connector 716 to a suction source. In some variations, the connector may include a stepped connector. The stepped connector may attach to a suction source, such that fluid and/or air trapped in the pleural space may be pulled through the frame 104 and to the suction source. In some variations, when not in use or connected to the plunger port 622, the check valve assembly may be attached to the frame 104 by a strap 726 so that it may then me readily available when needed to connect to the plunger port 622.

The frame 104 may further include an external valve port 610 on the Y-hub 718. In some variations, the external valve port 610 may be a luer activated valve. The luer activated valve may be connected to a lumen in the frame 104, which may then be connected to the balloon. In some examples, a syringe 724 may connect to the luer activated valve to supply air to the balloon.

In some variations, the rapid deployment chest port includes a stabilizing component configured to stabilize the frame inside and outside the chest cavity of a patient. The stabilizing component may be a single component configured to expand in the interior and exterior of the chest cavity of the patient. In some variations, the single stabilizing component is a balloon. In some examples, the stabilizing component includes a balloon attached to the outer diameter of the frame and an insertion stabilization platform attached to the outer diameter of the frame proximal to the balloon.

The insertion stabilization platform 606 may assist in securing the rapid deployment chest port in place. In a variation, the insertion stabilization platform may be a disc and may have a diameter sufficient to support and secure the rapid deployment chest port. In a variation, the insertion stabilization platform may have a diameter of 5 mm to 60 mm. In some variations, the insertion stabilization platform may have a diameter of at least 5 mm. In some variations, the insertion stabilization platform may have a diameter of at least 10 mm. In some variations, the insertion stabilization platform may have a diameter of at least 15 mm. In some variations, the insertion stabilization platform may have a diameter of at least 20 mm. In some variations, the insertion stabilization platform may have a diameter of at least 30 mm. In some variations, the insertion stabilization platform may have a diameter of at least 40 mm. In some variations, the insertion stabilization platform may have a diameter of at least 50 mm. In some variations, the insertion stabilization platform may have a diameter of less than 60 mm.

The insertion stabilization platform may be placed a distance from the needle and/or the bodily fluid inlet and provide an external surface for securing the placement of the rapid deployment chest port 700. In some variations, the insertion stabilization platform 606 may rest on the patient when the rapid deployment chest port is inserted the proper distance. The location of the insertion stabilization platform may be adjustable. The insertion stabilization platform may be initially placed at a distance from the needle and/or the bodily fluid inlet to mitigate the risk of injury to internal anatomy during insertion of the rapid deployment chest port. In some variations, the insertion stabilization platform may be located from 3 cm to 7 cm from the needle and/or the bodily fluid inlet. Most patients' pleural spaces are within 6.5 cm from the surface of the body, thus an initial spacing of the insertion stabilization platform of 6.5 cm may allow the rapid deployment chest port to clear the thickness of most patients while limiting insertion depth to prevent internal injury. In a variation, the insertion stabilization platform 606 may not be adjustable beyond 7 cm from the needle 102 and/or the bodily fluid inlet 102a.

In a variation, the insertion stabilization platform 606 may have one or more grooves or extrusions 602, such as a fixation flexure, to secure the insertion stabilization platform 606 to the frame. The insertion stabilization platform 606 may be integrated into the rapid deployment chest port 700 or be a separate piece through which the frame 104 and needle 102 and/or bodily fluid inlet 102a can be inserted.

In some variations, the insertion stabilization platform 606 is adjustable by way of the fixation flexure. In some examples, the insertion stabilization platform may include an opening to allow the insertion stabilization platform to be slid along the frame 104 and then secured into place against the patient using the fixation flexure after the rapid deployment chest port has been inserted the proper distance. The fixation flexure may have varying lengths and shapes to allow for ease of gripping and sliding the insertion stabilization platform. The fixation flexure may have two extensions, each with an outward extending flange. In some examples, the two extensions and outward extending flanges may be extended and curved. In some variations, the fixation flexure may be pinched to allow the insertion stabilization platform to slide along the frame, and release of the fixation flexure secures the insertion stabilization platform in place. In other variations, the extrusions may include a sliding-ratcheting mechanism for moving and securing the insertion stabilization platform to the frame.

In some variations, the slidable insertion stabilization platform 606 may be coupled to a pinch locking stabilizer 910 as depicted in FIG. 9C. A squeeze type pressure 912 and optionally 914 may be applied to pinch locking stabilizer 910 to move the pinch locking stabilizer along frame 104. When the pressure is released, pinch locking stabilizer 910 locks in place at a position along frame 104. Optionally, the pinch locking stabilizer 901 can be moved to a measured position based on distance markings 916 disposed on frame 104. The pinch locking stabilizer 910 can provide substantial advantages, as it is easy to manipulate rapidly under stressful circumstances, reducing the amount of time used to insert the device accurately.

In additional variations, the insertion stabilization platform 606 may form a seal around the frame to hold the insertion stabilization platform in place to limit initial insertion depth and prevent frame migration. For example, the insertion stabilization platform 606 may include a compression fitting. In some variations, the compression fitting may include a knob, compression sleeve, compression hub, and/or a compression pad. In some examples, the insertion stabilization platform 606 may include a threaded connection between the knob and compression hub. As the knob is tightened down onto the hub, the compression sleeve is compressed against the frame 104, preventing relative motion. In some variations, the insertion stabilization platform may include a balloon or pad on the patient facing surface, where the balloon or pad may be stationary or adjustable. In some variations, the insertion stabilization platform may have a coating of anesthetic or an anti-septic compound.

In some variations, the rapid deployment chest port may include a balloon 106 connected to the frame 104 near the needle 102. In some variations, the balloon is a compliant balloon. For example, the balloon may be a silicone balloon with a hardness of Shore A 50 or less.

The diameter of the balloon may range from about 5 mm to 55 mm. In some non-limiting variations, the balloon may have a diameter of at least 5 mm. In some non-limiting variations, the balloon may have a diameter of at least 21 mm. In some non-limiting variations, the balloon may have a diameter of at least 27 mm. In some non-limiting variations, the balloon may have a diameter of at least 38 mm. In some non-limiting variations, the balloon may have a diameter of at least 52 mm. In some non-limiting variations, the balloon may have a diameter of less than or equal to 55 mm. In some non-limiting variations, the balloon may have a diameter of less than or equal to 52 mm. In some non-limiting variations, the balloon may have a diameter of less than or equal to 38 mm. In some non-limiting variations, the balloon may have a diameter of less than or equal to 27 mm. In some non-limiting variations, the balloon may have a diameter of less than or equal to 21 mm. In some non-limiting variations, the balloon may have a diameter of less than or equal to 15 mm. In some non-limiting variations, the balloon may have a diameter of less than or equal to 10 mm.

The diameter of the insertion stabilization platform may be selected based on the diameter of the frame to limit damage to the patient during insertion and removal. The internal expanding may be large enough to provide sufficient force to prevent dislodgment or frame migration during the course of normal events is desirable while mitigating the risk that the rapid deployment chest port can damage tissue or otherwise harming the patient if the frame is exposed to uncommonly large forces. In at least some variations, the ratio between the diameter of the insertion stabilization platform and the diameter of the frame may range from 2.5 to 6. In one non-limiting variation, the ratio between the diameter of the insertion stabilization platform and the diameter of the frame is at least 2.5. In one non-limiting variation, the ratio between the diameter of the insertion stabilization platform and the diameter of the frame is at least 3.0. In one non-limiting variation, the ratio between the diameter of the insertion stabilization platform and the diameter of the frame is at least 4.0. In one non-limiting variation, the ratio between the diameter of the insertion stabilization platform and the diameter of the frame is at least 5.0. In one non-limiting variation, the ratio between the diameter of the insertion stabilization platform and the diameter of the frame is less than or equal to 6.0. In one non-limiting variation, the ratio between the diameter of the insertion stabilization platform and the diameter of the frame is less than or equal to 5.0. In one non-limiting variation, the ratio between the diameter of the insertion stabilization platform and the diameter of the frame is less than or equal to 4.0. In one non-limiting variation, the ratio between the diameter of the insertion stabilization platform and the diameter of the frame is less than or equal to 3.0. In at least one variation, the ratio between the diameter of the insertion stabilization platform and the diameter of the frame may range from 3 to 5. In an example, the ratio between the diameter of the insertion stabilization platform and the diameter of the frame may be 5.

In other non-limiting examples, the balloon may be made of Urethan, Pebax or any other thermoformed or extruded material. In a variation, the balloon may have a volume of 2 mL to 10 mL when used with a 16 French frame. The volume of the balloon, and thus the diameter of the balloon, may be adjusted based on the diameter of the frame based on the ratio of balloon to frame diameter. In a variation, the balloon may have a volume of 2 mL. In a variation, the balloon may have a volume of 3 mL. In a variation, the balloon may have a volume of 4 mL. In a variation, the balloon may have a volume of 5 mL. In a variation, the balloon may have a volume of 6 mL. In a variation, the balloon may have a volume of 7 mL. In a variation, the balloon may have a volume of 8 mL. In a variation, the balloon may have a volume of 9 mL. In a variation, the balloon may have a volume of 10 mL.

The external valve port 610 may be fluidly connected to the stabilizing component to expand and deflate the balloon and/or insertion stabilization platform. In a variation, the external valve port 610 may be fluidly connected to the balloon to facilitate the connection of a syringe to expand and deflate the balloon with air. The balloon may initially be deflated and against the outer diameter of the frame to aid in insertion of the rapid deployment chest port. In FIG. 3, the balloon 106 is shown in the deflated state against the frame 104. The balloon may then expand inside the pleural space to secure the rapid deployment chest port once it is properly in place. One or both of the balloon 106 and insertion stabilization platform 606 may serve to secure the rapid deployment chest port 700 in place in the patient's body.

The balloon and the insertion stabilization platform may be used in combination, on either side of the incision, to secure the rapid deployment chest port to the patient in the proper location. In some variations, the combination of the insertion stabilization platform and the balloon may allow the rapid deployment chest port to create an airtight seal between the inside and outside of the patient's body. This may allow for the efficient removal of air or fluid from the pleural space, reduce the risk of infection at the insertion site, and reduce the amount of time to treat the patient.

Referring now to FIG. 3, an alternative variation of the rapid deployment chest port 700 of FIG. 2 is shown. In this variation, the frame 104 may further include a peel away introducer 1202 for assisting in the insertion of the rapid deployment chest port 700. In some variations, the stabilizing component may include a compressed expandable portion of the frame that is compressed during insertion and expands after insertion to contour around internal and external tissue at an insertion site to prevent frame migration following deployment. In at least one example, the frame is compressed by the peel away introducer and then expands within the incision once the introducer is removed. The peel away introducer 1202 includes a heat-shrink material that compresses the frame onto the plunger. At the distal end of the rapid deployment chest port, the heat shrink may make a smooth transition from the plunger to the frame. In addition, the peel away introducer 1202 may include modeled finger grips that are used to peel the two halves of the heat-shrink material apart. In some variations, these finger grips may also be used to limit insertion depth. For example, the bottom of the finger grips may be used to mitigate the risk of injury to internal anatomy during insertion of the rapid deployment chest port by providing a stop to the insertion depth. In a variation, the distal end of the finger grips may be located from 3 cm to 7 cm from the needle and/or the bodily fluid inlet. In this example, the location of the finger grips of the peel away introducer may allow the rapid deployment chest port to clear the thickness of most patients while limiting insertion depth to prevent internal injury. In a variation, the finger grips of the peel away introducer may not be located beyond 7 cm from the needle 102 and/or the bodily fluid inlet 102a.

In an example method of removing air or fluid contained within a pleural space of a mammalian patient, a syringe device 100 as described herein may be employed with the rapid deployment chest port referenced above. The needle and/or bodily fluid inlet of the removable plunger and a distal portion of the frame may be inserted into a chest cavity of a patient. The balloon may then be expanded on the inside of the chest cavity of the patient. The plunger may be removed from the plunger port. The syringe device 100 may then be connected to the plunger port. Air or fluid may then be removed from the pleural space (e.g., using the syringe device 100 as described herein).

In an example method of repeating aspiration, a syringe device 100 as described herein may be employed. The syringe device 100 may be used, as described herein, to remove air or fluid may from the pleural space of a mammalian patient. The syringe device 100 may then be disconnected, the air or fluid may be removed and/or expunged from the syringe device 100, the syringe device 100 may be reconnected, and additional air or fluid may be removed from the pleural space using the syringe device 100 as described herein.

In variations, the syringe device 100 as described herein may be employed with the rapid deployment chest port referenced above in performing a method of repeating aspiration. The above-described method of removing air or fluid contained within a pleural space of a mammalian patient may be performed, including connecting the syringe device 100 to the rapid deployment chest port (e.g., the plunger port thereof) and removing air or fluid from the pleural space using the syringe device 100 as described herein. The syringe device 100 may then be disconnected from the rapid deployment chest port (e.g., the plunger port thereof). Disconnecting the syringe device 100 from the rapid deployment chest port (e.g., the plunger port thereof) may prevent or retard additional air or fluid from exiting while the syringe device 100 is disconnected. By way of non-limiting example, the plunger port may be a one-way valve configured to permit the passage of air or fluid only when the syringe device 100 is connected thereto. After the syringe device 100 is disconnected, the air or fluid may then be removed and/or expunged therefrom. Thereafter, aspiration may be repeated as many times as desired by reconnecting the syringe device 100 to the rapid deployment chest port (e.g., the plunger port thereof) and, once again, removing air or fluid from the pleural space using the syringe device 100 as described herein. The foregoing may advantageously provide a quicker, more efficient, and more reliable means to measure an amount of removed fluid and, thus, a necessary amount of replacement fluid.

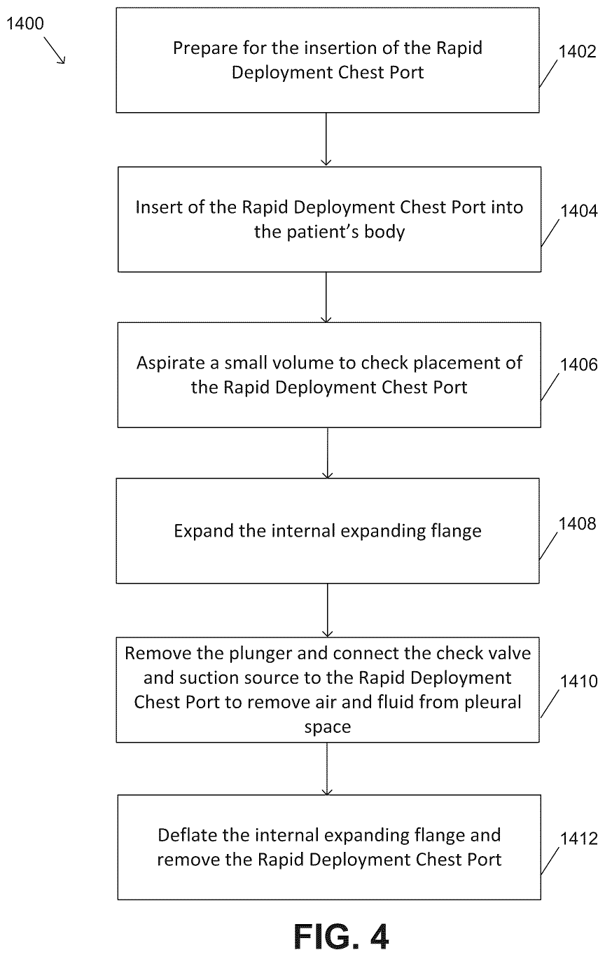

Referring now to FIG. 4, an exemplary variation of a method for accessing the pleural space of a patient 1400 is shown. The patient's pleural space may need to be accessed urgently or non-urgently. Non-limiting treatments or needs for accessing the pleural space include treatment of tension pneumothorax, treatment of non-tension pneumothorax, removal of fluid from trauma, drainage of a small amount of fluid, and/or administration medication to the pleural space. At optional step 1402, preliminary steps are taken to prepare for the insertion of the rapid deployment chest port. These steps may include adjusting the location of the insertion stabilization platform along the frame or preparing a patient's body. In exemplary variations, the depth of insertion is a function of the distance between the needle and/or bodily fluid inlet, which leads the insertion into the patient's body, and the insertion stabilization platform, which stops the insertion when it comes to rest against the patient's body. Additionally, depending upon the patient, too shallow an insertion may be ineffective; too deep an insertion may cause undue harm. Other preliminary steps, such as sanitizing the needle, may be required in some variations or situations; however, in exemplary variations, the rapid deployment chest port is stored in sterile, self-contained packaging designed for rapid deployment, and the rapid deployment chest port itself may be coated in one or more of: a disinfectant, antiseptic fluid, or anesthetic. Accordingly, in exemplary variations, optional step 1402 will be minimal, if at all.

At step 1404, the rapid deployment chest port is inserted into the patient's body. In some variations, this may include inserting the needle and/or the bodily fluid inlet of the plunger and distal portion of the frame of the rapid deployment chest port into the patient's chest cavity. Due to the durability of the thoracic cavity, this insertion may require considerable force. In some variations, it may be desirable to access the pleural space indirectly, such as through the patient's axilla. In exemplary variations, the insertion is complete when the insertion stabilization platform rests against the patient's body.

At optional step 1406, an aspiration syringe device (e.g., aspiration syringe device 100) connected to the handle is used to aspirate a small volume from the pleural space to confirm the rapid deployment chest port is inserted to the correct depth. This step may further include adjusting the depth of the rapid deployment chest port, if necessary. Optional step 1406 may occur simultaneously with step 1404.

At step 1408, the stabilizing component is expanded in at least the inside of the patient's chest cavity. In some variations, the stabilizing component is the balloon. In some variations, the balloon is expanded by filling it with air through a syringe connected to the external valve port on the frame. In some variations, step 1408 may optionally include sliding or locking the insertion stabilization platform such that it rests on the patient's chest. At the conclusion of step 1408, the rapid deployment chest port may be securely set in the patient at the proper insertion depth for the patient.

At step 1410, plunger 704 may be removed from the frame and a check valve, by way of a luer connector, may be connected to a 1-way valve at the proximal end of the frame. In this example, a stepped connector connected to the proximal end of the check valve may be connected to a suction source to remove air or fluid from the pleural space.

Finally, at step 1412, the stabilization component, such as the balloon, is deflated prior to removal of the rapid deployment chest port. In some examples, this may include withdrawing air from the balloon using the syringe attached to the external valve port. The rapid deployment chest port may then be safely removed from the patient's body.

A number of variations of the disclosure have been described. While this specification contains many specific implementation details, there should not be construed as limitations on the scope of any disclosures or of what may be claimed, but rather as descriptions of features specific to particular variations of the disclosure. While variations of the disclosure are described herein by way of example using the illustrative drawing, those skilled in the art will recognize the disclosure is not limited to the variations or drawing described. It should be understood the drawing and the detailed description thereto are not intended to limit the disclosure to the form disclosed, but to the contrary, the disclosure is to cover all modifications, equivalents, and alternatives falling within the spirit and scope of variations of the disclosure as defined by the appended claims.

The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include”, “including”, and “includes” mean including but not limited to. To facilitate understanding, like reference numerals have been used, where possible, to designate like elements common to the figures.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted the terms “comprising”, “including”, and “having” can be used interchangeably.

Certain features that are described in this specification in the context of separate variations can also be implemented in combination in a single variation. Conversely, various features that are described in the context of a single variation can also be implemented in combination in multiple variations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while method steps may be described in a particular order, this should not necessarily be understood as requiring that such operations be performed in the particular order shown or in a sequential order, or that all illustrated operations be performed, to achieve desirable results.

1. An aspiration syringe device, comprising:
an elongated housing having an open proximal end and an open distal end;
a plunger having a proximal end and a distal end and configured for axial sliding within the housing, the proximal end of the plunger occludably received through the proximal end of the housing;
a compression spring disposed about a portion of the plunger external to the housing, the compression spring configured to urge the distal end of the plunger away from the proximal end of the housing; and
a retention mechanism configured to fixedly engage with the plunger so as to retain the plunger in position relative to the housing with the compression spring in an at least partially compressed state.

2. The aspiration syringe device of claim 1, wherein removal of the retention mechanism from fixed engagement with the plunger causes the compression spring to transition to an uncompressed state and urge the distal end of the plunger away from the proximal end of the housing.

3. The aspiration syringe device of claim 1, wherein the retention mechanism is configured to fixedly engage with the plunger adjacent the distal end of the plunger.

4. The aspiration syringe device of claim 3, wherein the retention mechanism passes through an opening defined in the housing.

5. The aspiration syringe device of claim 4, wherein the opening is defined in a side wall of the housing and adjacent the proximal end of the housing.

6. The aspiration syringe device of claim 4, wherein the retention mechanism is a pin having a head and a shaft extending outwardly therefrom, the shaft configured to be received through the opening defined in the housing and to engage with the plunger.

7. The aspiration syringe device of claim 1, wherein the compression spring is disposed in circumferential relationship about the portion of the plunger external to the housing and extends from the distal end of the plunger toward the proximal end of the plunger.

8. The aspiration syringe device of claim 7, wherein the compression spring has a first end and a second end, the first end of the compression spring abutting a lateral extension at the proximal end of the housing and the second end of the compression spring abutting a flange portion at the distal end of the plunger.

9. The aspiration syringe device of claim 1, wherein the distal end of the housing includes a luer connector.

10. The aspiration syringe device of claim 1, wherein the plunger includes a retention tab projecting radially outward therefrom, the retention tab configured to engage with the retention mechanism so as to retain the plunger in position relative to the housing with the compression spring in the at least partially compressed state.

11. The aspiration syringe device of claim 1, wherein the plunger includes a compressible stopper at the proximal end thereof, the stopper configured to provide a wiping action along an inner wall of the housing and to prevent the plunger from being completely removed from the housing.

12. The aspiration syringe device of claim 1, wherein the housing has a volume of at least 100 mL.

13. The aspiration syringe device of claim 12, wherein the housing has a volume of at least 300 mL.

14. The aspiration syringe device of claim 1, wherein the compression spring has a preselected tension configured to provide a desired suction pressure for the aspiration syringe device.

15. The aspiration syringe device of claim 14, wherein the compression spring has a preselected tension configured to provide a suction pressure for the aspiration syringe device of from about −10 cmH₂O to about −40 cmH₂O.

16. A method of determining a required amount of replacement blood for a mammalian patient using the aspiration syringe device of claim 1, the method comprising:
removing the retention mechanism from fixed engagement with the plunger, thereby causing the compression spring to transition to an uncompressed state and urge the distal end of the plunger away from the proximal end of the housing;
continuously removing blood contained within the mammalian patient as the compression spring transitions to the uncompressed state;
determining an amount of blood removed from the mammalian patient by referencing an amount of blood contained within the housing after the compression spring has transitioned to the uncompressed state; and
calculating a required amount of replacement blood as corresponding to the amount of removed blood contained within the housing.

17. The method of claim 16, further comprising:
replacing the blood removed from the mammalian patient with the calculated required amount of replacement blood.

18. A method of measuring a volume of fluid loss during aspiration using the aspiration syringe device of claim 1, the method comprising:
removing the retention mechanism from fixed engagement with the plunger, thereby causing the compression spring to transition to an uncompressed state and urge the distal end of the plunger away from the proximal end of the housing;
continuously removing fluid contained within the mammalian patient as the compression spring transitions to the uncompressed state;
determining an amount of fluid removed from the mammalian patient by referencing an amount of removed fluid contained within the housing after the compression spring has transitioned to the uncompressed state; and
calculating a required amount of replacement fluid as corresponding to the amount of removed fluid contained within the housing.

19. The method of claim 18, further comprising:
replacing the fluid removed from the mammalian patient with the calculated required amount of replacement fluid.

20. (canceled)

21. A rapid deployment chest port, comprising:
a frame comprising a lumen and a plunger port;
a removable plunger defining a bodily fluid inlet at the distal end, wherein the removable plunger is within the lumen of the frame;
a stabilizing component configured to stabilize the frame inside and outside a chest cavity of a patient; and
the aspiration syringe device of claim 1 fluidly connected to the plunger port.

22.-26. (canceled)
